(19) United States
(12) Patent Application Publication
(10) Pub. No.: US 2004/0092549 A1
(43) Pub. Date: May 13, 2004

(54) Methods and compositions for selective cancer chemotherapy

(76) Inventor: Raxit J Jariwalla, Saratoga, CA (US)

Correspondence Address: Drummond & Duckworth, 5000 Birch Street, Suite 440, East Tower, Newport Beach, CA, 92660, US

(21) Appl. No.: 09/830,912

(22) PCT Filed: Aug. 30, 1999
(86) PCT No.: PCT/US99/19449

(51) Int. Cl. A61K 31/47; A61K 31/555

(52) U.S. Cl. 514/310; 514/185

(57) ABSTRACT

A selective chemotherapy method includes the step of contacting tumor cells with a mineral ascorbate/vitamin C metabolite composition. A chemotherapeutic composition comprises the mineral ascorbate/vitamin C metabolite composition in a pharmacologically acceptable intravenous carrier.

### FIELD OF THE INVENTION

This invention relates to tumor-cytotoxic chemotherapeutic methods.

In another aspect the invention relates to tumor-cytotoxic chemotherapeutic compositions.

More particularly the invention concerns tumor-cytotoxic chemotherapeutic methods and compositions for treating cancers in a human host.

### BACKGROUND OF THE INVENTION

Tumor cytotoxic chemotherapeutic agents preferentially induce death (apoptosis) of malignant cells. Because of similarities between normal and malignant cells, both being born of the same host, a chemotherapeutic dose which induces apoptosis of tumor cells can also be toxic to normal cells. In order to effect a remission, the tumor-cytotoxic agent must often push the limits of acceptable side effects. Ideally, the tumor-cytotoxic agent should be “selective”, i.e., there should be a large gap between the lower dose required to induce tumor cell death, for efficacy as a tumor-cytotoxic chemotherapeutic agent, and the higher dose which is toxic to the patient's normal cells.

The adverse side-effects of chemotherapy may include hair loss, nausea and vomiting, cardiac toxicity and secondary cancers. One of the most common side-effect toxic manifestations of many cytotoxic agents is bone marrow suppression, which can lead to immune suppression and hematopoietic dysfunctions. Because infectious complications are one of the major causes of death in cancer patients, it would be highly desirable to provide non-toxic tumor-cytotoxic chemotherapeutic compositions and methods without immunosuppressive side effects.

Compounds having vitamin C activity, e.g., ascorbic acid and ascorbate derivatives, are not immunosuppressive, but are effective intravenous cytotoxic chemotherapeutic agents against a wide variety of cancers. Riordan et al., Medical Hypotheses, 1995, 44, 207-213. However, there is no vitamin C storage mechanism in human tissues and it is all metabolized and/or excreted. Further, because of gastrointestinal complications, it is difficult to establish and maintain high serum levels of vitamin C by oral administration of ascorbic acid. Thus, it is generally considered necessary to administer ascorbic acid intravenously in order to establish and maintain plasma levels sufficiently high to achieve cytotoxicity. Therefore, it would be extremely advantageous to provide tumor chemotherapeutic compositions, containing forms of vitamin C other than ascorbic acid, which can be orally administered in doses sufficiently high to establish and maintain a tumor cytotoxic level of serum vitamin C.

However, because even vitamin C can be toxic to normal human cells if the plasma concentration is sufficiently high, it would also be highly desirable to provide selective vitamin C tumor chemotherapeutic compositions in oral or intravenous dosage forms, which achieve tumor cell apoptosis at lower plasma concentrations than those required for ascorbic acid to induce tumor cell apoptosis. Because the tumor cytotoxic concentration of vitamin C administered from such dosage forms would be lower, it would be more feasible to establish and maintain a chemotherapeutically effective plasma concentration at a level which would be below the vitamin C plasma apoptosis level for normal cells.

### THE PRIOR ART

As reviewed by Cameron et al. (Cancer Res., 39:663-81 (1979)) some clinical trials have shown significant increases in survival times of cancer patients receiving vitamin C.

Elvin et al. (Eur. J. Cancer Clin. Oncol. 17(7):759-65 (1981)) reported that adducts of ascorbic acid with aldehydes such as methylglyoxal and acetylacrolein inhibit growth of Ehrlich ascites carcinoma in mice.

EP-A-0086544 proposes uses of ketals and acetals of ascorbic acid as angiogenesis-inhibiting agents. (Angiogenesis refers to the process of new blood vessel development, the proliferation of new blood vessels being involved in tumor growth.)

EP-A-0148094 and U.S. Pat. 5,032,610 propose that orally administered or intravenously administered 5,6-O-benzylidene-L-ascorbic acid and salts thereof and mixtures thereof with L-ascorbic acid and salts thereof exhibit anti-cancer properties.

Concomitant administration of 3-amino-1,2,4-triazole enhances the cytotoxicity of ascorbic acid to Ehrlich ascites tumor cells and the addition of vitamin K3 (menadione sodium bisulfite) appears to increase preferential tumor cytotoxicity of ascorbic acid. Benande et al., Oncology, 23:33-43 (1969).

Also, prior workers have shown that catalytic concentrations of Cu²⁺ increased the preferential toxicity of ascorbic acid for several malignant melanoma cell lines, including four human-derived lines. Bram et al., Nature 284: 629-631(1980).

Several leukemic, pre-leukemic and myeloma progenitor cells derived from human patients were reported to be sensitive to ascorbic acid concentrations attainable in vivo, without any toxicity to normal hemopoietic cells. Park et al., Cancer Res. 4:1062-65 (1980); Am.J.Clin.Nutr. 54:1241S-46S (1991).

### DESCRIPTION OF THE DRAWINGS

FIG. 1 is a bar graph which illustrates the apoptosis of various tumor cell-types by the “mineral ascorbate plus metabolites” composition employed in the practice of the preferred invention, as illustrated by Test 1.

FIG. 2 is a similar bar graph which illustrates the selectivity of apoptosis of various tumor cell-types over normal cells by the “mineral ascorbate plus metabolites” composition employed in the preferred practice of the invention, as illustrated by Test 1.

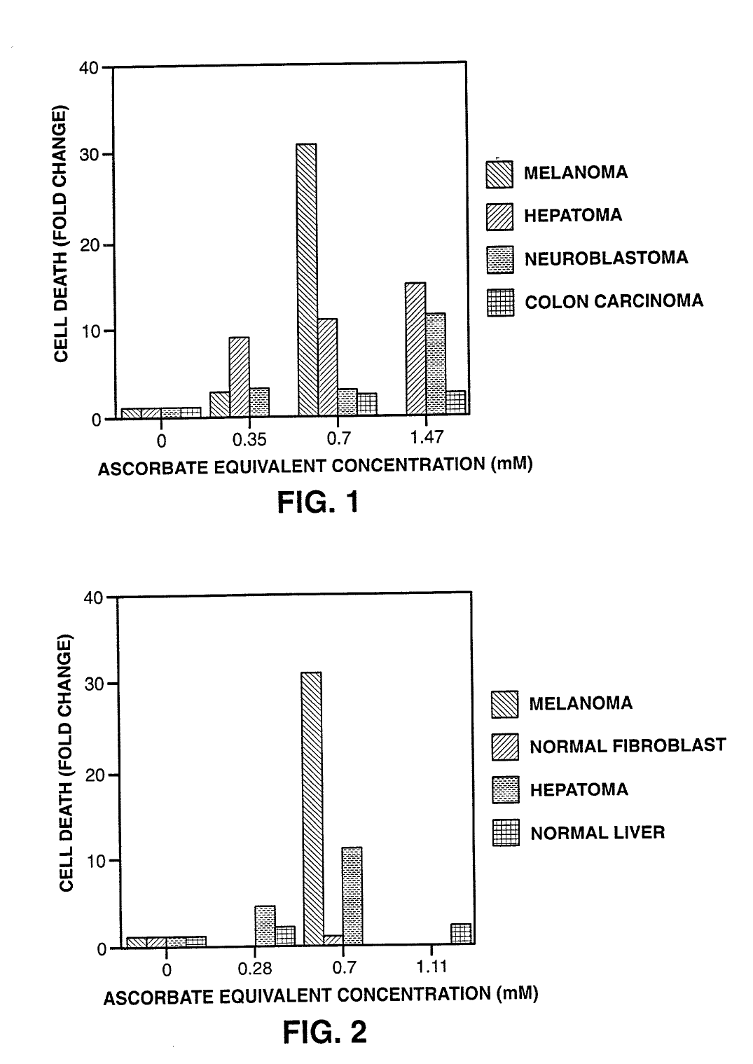

### BRIEF DESCRIPTION OF THE INVENTION

The chemotherapy method of the present invention includes the step of contacting tumor cells with a composition comprising a plasma-soluble mineral ascorbate and one or more vitamin C metabolites selected from the group consisting of aldonic acids, the aldono-lactones, aldono-lactides and non-toxic metal salts of aldonic acids, dehydroascorbic acid, threose, erythreose, 4-hydroxy-5-methyl-3(2H)-furanone, 3-hydroxykojic acid and 5-hydroxymaltol.

The novel chemotherapeutic compositions of the invention, which are useful in practicing the method of the invention, comprises the components of such chemotherapeutic compositions in a pharmacologically acceptable intravenous carrier.

### THE PREFERRED EMBODIMENTS OF THE INVENTION

The components of the above-described chemotherapeutic composition are simply mixed together in appropriate proportions. The exact proportions are not highly critical. Operable and optimum proportions can be determined and varied within limits which can be determined without undue experimentation by those skilled in the art, e.g., by employing in vitro tests such as those described below. Alternatively, in accordance with the presently preferred embodiment of the invention, suitable mineral ascorbate-metabolite compositions, containing these components in appropriate proportions, are commercially available under the registered trademark ESTER-C® from Inter-Cal Corporation, Prescott, Ariz., USA. These compositions are further described in U.S. Pat. Nos. 4,822,816; 4,968,716; and 5,070,085, incorporated herein by reference.

The cytotoxically effective vitamin C plasma concentration provided by the chemotherapeutic methods and compositions of the invention will vary according to the specific type of tumor cells being treated and can be determined by in vitro tests such as those described below, animal tests and human in vivo trials, in accordance with art-recognized techniques.

The chemotherapeutic compositions of the invention are formulated for intravenous administration by inclusion of the mineral ascorbate and vitamin C metabolite components in a pharmaceutically acceptable intravenous carrier, i.e., a sterile, non-toxic solution of the components in a carrier, formulated to provide appropriate osmolality, pH, etc., in accordance with art-recognized techniques. For example, Ringer's Lactate is an appropriate intravenous carrier.

The concentration of vitamin C in the intravenous carrier can be varied within wide limits to suit the requirements of treatment. For example, when it is desired to establish an ascorbic acid equivalent plasma concentration in the range 150-200 mg/dL, an appropriate dosage for an 8-hour, 1000 cc infusion is 100-150 mg of ascorbate provided by the mineral salt. It may be required to repeat such infusions several times before reaching and maintaining the desired plasma concentration, depending on the capacity of the patient's system for ascorbate destruction, elimination or excretion.

It is also possible to employ oral dosage forms containing the mineral ascorbate/vitamin C metabolite compositions to establish initial plasma concentrations of these compositions which are effective to induce apoptosis in some forms of tumors. According to my present information, at oral dosages in the range of approximately 12-15 grams of ascorbate per day, a plasma level (AA equivalent) of approximately 5 mg/dl is attainable, which is sufficient to induce selective apoptosis of melanoma and hepatoma cells.

Moreover, once a selective tumor apoptosis-inducing plasma concentration is obtained by intravenous administration, that concentration can be maintained by administration of oral dosage forms or by a combination of oral and intravenous administration.

### EXAMPLES

The following examples are presented for the purpose of illustrating the practice of the invention and identifying the presently preferred embodiments thereof to persons skilled in the art, and are not to be construed as limitations on the scope of the invention.

Several tumor cell lines and corresponding normal non-malignant cell lines are tested for apoptosis by Ester-C® (Calcium Ascorbate plus metabolites) versus four other test compositions, calcium ascorbate (CA) alone, calcium threonate (CT) alone and calcium ascorbate plus calcium threonate (CA+CT) and sterile water (sH₂O).

### Example 1

### Test Procedures

The cell lines are:

Malme-3M Melanoma, Human (ATCC No. HTB-64)

Malme-3 Normal Human Skin Fibroblasts (ATCC No. HTB-102)

SK-Hep-1 Liver adenocarcinoma, Human (ATCC No. HTB-52)

WRL Normal Human Liver Cells (ATCC No. CL-98)

SK-N-MC Neuroblastoma, Human (ATCC No. HTB-10)

T-84 Colon Carcinoma, Human (ATCC No. CCL-248)

Stock cells are grown in the growth media, as follows:

| Cell Line | Growth Media |
|---|---|
| SK-Hep-1, SK-N-MC and | Eagle's Minimal Essential |
| WRL 88 | Medium in Earle's salts |
| | supplemented with 2 mM L- |
| | glutamine , 1 mM sodium |
| | pyruvate, 10% fetal bovine |
| | serum (FBS) and antibiotics |
| | (penicillin, streptomycin, |
| | amphotericin B) |
| Malme-3 and Malme 3-M | McCoy's medium with L- |
| | glutamine, 15% FBS and |
| | antibiotics |
| T-84 | 1:1 mixture of Dulbecco's |
| | modified MEM and Ham's F-12 |
| | medium with L-Glutamine, |
| | pyridoxal hydrochloride, 25 mM |
| | Hepes plus 5% FBS and |
| | antibiotics |

All cultures are maintained at 37 C in a humidified atmosphere of 5%CO₂/95% air. Media and culture reagents are obtained from Life Technologies (Gibco/BRL, Long Island, N.Y.). FBS is obtained from Hyclone Labs (Logan, Utah).

Test materials are obtained from Inter-Cal Corporation (Prescott, Ariz.), as follows:

| | |
|---|---|
| Test 1 | Ester-C ® Mineral Ascorbate (see below) |
| Test 2 | Calcium Ascorbate (USP Grade), 82.15% |
| | ascorbic acid (AA) equivalent |
| Test 3 | Calcium Threonate, 87.08% L-threonic |
| | acid (TA) equivalent |
| Test 4 | Calcium Ascorbate (U.S.P.) + Calcium |
| | U.S.P., 81.21% AA, 1% TA equivalent |
| Test 5 | Ascorbic Acid |

The Test 1 material contains the following by laboratory analysis:

| | |
|---|---|
| Calcium Ascorbate | 78.4% AA equivalent |
| Calcium Threonate | .9% TA equivalent |
| Other AA Metabolites¹ | 10.4% AA equivalent |
| Water of Crystallization | Balance |

¹aldonic acids, the aldono-lactones, aldono-lactides and non-toxic metal salts of aldonic acids, dehydroascorbic acid, threose, erythreose, 4-hydroxy-5-methyl-3(2H)-furanone, 3-hydroxykojic acid and 5-hydroxymaltol.

Ascorbic Acid (tissue culture grade) is obtained from Sigma Chemical Co. (St. Louis, Mo.). Control compositions consist of growth medium, Ringer's Solution or sterile water, as appropriate.

All working solutions are prepared from master stocks immediately before use. A 60 mM master stock solution of AA is prepared in serum-free growth medium and stored at −15 C. Working solutions are made from 10× strength stock solutions by dilution in growth medium. A 30 mM (1gm/%) stock of calcium threonate is made in Ringer's solution (Fay and Verlangieri, Life Sciences, 49:1377 (1991)) or warm sterile water. Working solutions are made as 1× strength stock (in Ringer's solution) or as 10× stock (in sH₂O), depending on the nature of the treatment, i.e., short-term versus continuous, (see below).

For evaluation of the Test 1 material, 1-1.3% master stock solution of the Test material is prepared in warm sterile water. Working stock solutions (10× strength) are made in sterile water immediately before use. For comparative evaluation, stock solutions of Test 2 and Test 4 solutions are prepared in sterile water, normalized to contain AA equivalents identical to the Test 1 stock. These stocks are stored at room temperature for use in evaluations.

### Example 2

### Treatment of Cell Cultures with Ascorbic Acid and/or Calcium Threonate

0.25-1.0×10⁵ cells of tumor-derived or normal liver cell lines are seeded and cultured in individual wells of a 24-well cluster plate in the presence of increasing concentrations of freshly prepared supplement consisting of ascorbic acid (AA) or calcium ascorbate (CA). Cultures are re-fed periodically with additions of respective supplements, with or without medium change as indicated. Controls consist of cells receiving growth medium without added supplement.

For treatment with calcium threonate (CT), cells are allowed to attach by overnight incubation. The following day, threonate treatment is initiated using one of two protocols.

In one protocol (short exposure), monolayers are washed and exposed directly to 1 mL/well of 7.5-30 mM threonate (prepared in Ringer's solution) for brief periods at 37 C. as described by Fay and Verlangieri (referenced above). Controls are exposed to 1 mL/well of Ringer's solution alone for similar intervals. After exposure, the solution is removed and replaced by growth medium.

In the other protocol (continuous exposure), one-tenth volume of 10× strength threonate (prepared in sterile water) is added to the culture medium and the incubation continued at 37 C. This same treatment is repeated by daily additions of fresh working solution.

### Example 3

### Treatment of Cell Cultures With Ascorbic Acid Plus Calcium Threonate

Cells are treated with calcium threonate (CT), as in Example 2, for sixty minutes at 37C followed by addition of ascorbic acid (AA) in Ringer's solution and continuous incubation for thirty minutes. At the end of the treatment period, the solution is removed and replaced by 1 mL/well of growth medium.

### Example 4

### Treatment of Cell Cultures with Test 1 (Ester-C® Calcium Ascorbate Plus Metabolites) and Test 4 (Calcium Ascorbate Plus Calcium Threonate)

Subconfluent monolayers of cell cultures, seeded in 24-well cluster plates, are supplemented with one-tenth volume of working stock solutions to obtain final Test 1 concentration in the range 0.006 to 0.06% (corresponding to 0.28 mM-2.8mM ascorbic acid equivalents). Parallel sets of wells are treated similarly with stock solutions of CA+CT and CA alone, containing the same equivalents of AA as Test 1. Control cultures are treated with an equivalent volume of sterile water. Periodic treatment with these compositions is repeated at 1-2 day intervals by direct addition of fresh solutions (without change of growth medium).

### Example 5

### Assay of Cell Survival and Cell Death (Apopotosis)

Cell survival following treatment is assessed at predetermined intervals by taking viable cell counts using a Neubauer haemocytometer. Viable cells are scored as those capable excluding trypan blue as previously described (Harakeh and Jariwalla, Am.J.Clin.Nutr. 54:1231S-1235S (1991)). The data are used to plot viable cell culture (# of cells/ml) against the concentration of Test solutions to evaluate the effect on cell survival.

The induction of apoptosis following treatment of various tumor cell types with the Test compositions and controls described in Example 1 is evaluated using an enzyme-linked immunoassay (“ELISA”) developed by Boehringer-Mannheim (Indianapolis, Ind.). This assay specifically screens and detects histone-associated DNA complexes (nucleosomal fragments) appearing in the cytoplasm of treated cells relative to that in untreated controls. The presence and level of nucleosomal fragments in cytoplasmic lysates after different treatments is determined using the procedure specified in the cell-death detection ELISA kit, supplied by Boehringer-Mannheim.

Briefly, the ELISA assay is carried out as follows. At different intervals following treatment with the Test compositions, medium is aspirated and cell membranes are lysed by incubation with 200-500 μL of lysis solution for 30 minutes at room temperature. Cell lysate is collected in an Eppendorf tube and centrifuged at 2500 rpm for 10 minutes to separate the nuclear fraction. An aliquot of the supernatant containing the cytoplasmic fraction is used to quantify the nucleosomal fragments by photometric detection at 410 nm in a micro plate reader. Data are processed as follows: The mean absorbance at 410 nm is plotted against the concentration of the Test compound to compare the relative apoptosis-inducing doses determined for each treatment and compared. The minimal apoptosis-inducing dose is defined as that required to cause 2-fold change in apoptosis (i.e., nucleoprotein level) over that in the untreated control. Maximal apoptosis is defined as the maximal fold change in apoptosis over control.

TABLE 1
SUMMARY OF DATA

| | | MINIMUM APOPTOTIC | | MAXIMUM FOLD INCREASE | | |
|---|---|---|---|---|---|---|
| | | DOSE | | IN APOPTOSIS | | MAXIMUM FOLD |
| | CELL | DOSE | # OF | DOSE | # OF | INCREASE |
| COMPOSITION | LINE | CONC. (%) | TREATMENTS | CONC. (%) | TREATMENTS | IN APOPTOSIS |
| [Calcium | Malme-3 | 0.025 | 2 | 0.033 | 2 | 3.58 |
| Ascorbate + | Malme-3M | 0.006 | 1 | 0.025 | 2 | 116 |
| Metabolites] | WRL-68 | 0.006-0.012 | 3 | 0.025 | 2 | 2.04 |
| | SK-Hep-1 | 0.006 | 3 | 0.033 | 3 | 14.9 |
| | SK-N-MC | 0.008 | 2 | 0.033 | 4 | 13.6 |
| | T-84 | 0.015 | 3 | 0.02-0.03 | 4 | 4 |
| [Calcium | Malme-3 | 0.025 | 2 | 0.025 | 2 | 2.16 |
| Ascorbate + | Malme-3M | 0.012 | 2 | 0.025 | 2 | 65.7 |
| Calcium | WRL-68 | >0.05 | 3 | 0.0125 | 2 | 1.68 |
| Threonate] | SK-Hep- | 0.006 | 3 | 0.033 | 3 | 9.31 |
| | SK-N-MC | 0.015 | 4 | 0.033 | 4 | 7.02 |
| | T-84 | >0.06 | 3 | 0.033 | 3 | 1.5 |
| Calcium | Malme-3 | 0.025 | 2 | 0.025 | 2 | 2.16 |
| Ascorbate | Malme-3M | 0.012 | 2 | 0.025 | 2 | 92.8 |
| | WRL-68 | 0.012 | 3 | 0.012 | 3 | 2.22 |
| | SK-Hep-1 | 0.006 | 3 | 0.033 | 3 | 12.04 |
| | SK-N-MC | 0.015 | 4 | 0.015 | 4 | 5.03 |
| | T-84 | >0.06 | 3 | 0.033 | 3 | 1.77 |

In Table 1, above, the “Minimum Apoptotic Dose” is the concentration of the Composition required to cause a 2-fold change in apoptosis over the control. The “Number of Treatments” is the total number of treatments with the Composition. The “Maximum Fold Increase in Apoptosis” is the maximal fold change in apoptosis over the control and the “Maximum Dose” is the concentration of the Composition which induces the “Maximum Fold Apoptosis” compared to the control.

### CONCLUSIONS

The results of tests described above lead to the following conclusions:

The mineral ascorbate/vitamin C metabolite compositions illustrated by the Test 1 composition induce selective cell death (apoptosis) of diverse tumor cell-types in a dose-dependent fashion.

Mineral ascorbate/vitamin C metobolite compositions (as illustrated by the Test 1 composition) achieve apoptosis, i.e., minimum two-fold increase in cell death rate, at lower concentrations (AA equivalent) than is required to achieve such decrease with either mineral ascorbate alone (as illustrated by the Test 2 composition) or with ascorbic acid alone.

The maximal level of apoptosis achievable with mineral ascorbate/vitamin C metabolite compositions (as illustrated by the Test 1 composition) against specific cell types, is higher than achievable with mineral ascorbate or ascorbic acid alone.

The AA equivalent concentration of mineral ascorbate/vitamin C metabolite compositions (as illustrated by the Test 1 composition) required to induce apoptosis in tumor cells is lower than for normal cells, and the magnitude of cell death in normal cells is considerably smaller than in tumor cells.

Treatment of cell cultures with ascorbic acid (AA) and/or calcium threonate (CT) (as illustrated in Example 2) produced selective dose-dependent cell death (apoptosis) in hepatoma and melanoma cells as compared to their respective normal cellular counterparts.

Pretreatment of hepatoma cells with CT followed by application of AA induced higher level of cellular apoptosis than corresponding dose of AA or CT alone.

Having described the invention in such terms as to enable those skilled in the art to understand and practice it, and, having described the presently preferred embodiments thereof, I claim:

1. A selective chemotherapy method which includes the step of contacting tumor cells with a composition comprising:
(a) a plasma-soluble metal salt of ascorbic acid; and
(b) one or more vitamin C metabolites selected from the group consisting of
(i) aldonic acids, and the aldono-lactones, aldono-lactides and non-toxic metal salts thereof, and
(ii) dehydroascorbic acid, threose, erythreose, 4-hydroxy-5-methyl-3 (2H )-furanone, 3-hydroxykojic acid and 5-hydroxymaltol.

2. A composition comprising the chemotherapeutic composition of claim 1 in a pharmacologically acceptable intravenous carrier.
